(19) United States
(12) Patent Application Publication
(10) Pub. No.: US 2014/0058365 A1
(43) Pub. Date: Feb. 27, 2014

(54) System and Method for Using Compensating Incisions in Intrastromal Refractive Surgery

(71) Applicants: Josef F. Bille, Heidelberg (DE); Luis Antonio Ruiz, Bogota (CO)

(72) Inventors: Josef F. Bille, Heidelberg (DE); Luis Antonio Ruiz, Bogota (CO)

(21) Appl. No.: 13/854,826

(22) Filed: Apr. 1, 2013

Related U.S. Application Data
(63) Continuation-in-part of application No. 12/780,180, filed on May 14, 2010, now Pat. No. 8,409,179.
Application No. 12/780,180 is a continuation-in-part of application No. 12/757,798, filed on Apr. 9, 2010.
Application No. 12/757,798 is a continuation-in-part of application No. 12/105,195, filed on Apr. 17, 2008, now Pat. No. 7,717,908.
Application No. 12/105,195 is a continuation-in-part of application No. 11/958,202, filed on Dec. 17, 2007, now Pat. No. 7,717,907.

(51) Int. Cl. A61F 9/008

(52) U.S. Cl. CPC A61F 9/00827
USPC 606/5

(57) ABSTRACT

A system and method for performing intrastromal ophthalmic laser surgery requires Laser Induced Optical Breakdown (LIOB) of stromal tissue without compromising Bowman's capsule (membrane). In detail, at least one singularly unique, intrastromal compensating incision is made relative to a defined axis of the eye. The location of this compensating incision is specifically selected to counter and minimize the adverse effect on vision that may be caused by a predetermined (e.g. surgically introduced) asymmetrical optical condition.

This application is a continuation-in-part of application Ser. No. 12/780,180 filed May 14, 2010, which is currently pending, and which is a continuation-in-part of application Ser. No. 12/757,798 filed Apr. 9, 2010, which is currently pending, and which is a continuation-in-part of application Ser. No. 12/105,195 filed Apr. 17, 2008, which issued as U.S. Pat. No. 7,717,908, and which is a continuation-in-part of application Ser. No. 11/958,202 filed Dec. 17, 2007, which issued as U.S. Pat. No. 7,717,907. The contents of application Ser. Nos. 12/780,180 and 12/757,798, and U.S. Pat. Nos. 7,717,908 and 7,717,907 are incorporated herein by reference.

### FIELD OF THE INVENTION

The present invention pertains generally to methods for performing intrastromal ophthalmic laser surgery. More particularly, the present invention pertains to laser surgery wherein stromal tissue is cut with a singularly unique incision. The present invention is particularly, but not exclusively, useful as a method for performing intrastromal ophthalmic laser surgery wherein reshaping of the cornea is accomplished by inducing a redistribution of bio-mechanical forces in the cornea to correct a predetermined asymmetrical optical condition.

### BACKGROUND OF THE INVENTION

The cornea of an eye has five (5) different identifiable layers of tissue. Proceeding in a posterior direction from the anterior surface of the cornea, these layers are: the epithelium; Bowman's capsule (membrane); the stroma; Descemet's membrane; and the endothelium. Behind the cornea is an aqueous-containing space called the anterior chamber. Importantly, pressure from the aqueous in the anterior chamber acts on the cornea with bio-mechanical consequences. Specifically, the aqueous in the anterior chamber of the eye exerts an intraocular pressure against the cornea. This creates stresses and strains that place the cornea under tension.

Structurally, the cornea of the eye has a thickness (T), that extends between the epithelium and the endothelium. Typically, “T” is approximately five hundred microns (T=500 μM). From a bio-mechanical perspective, Bowman's capsule and the stroma are the most important layers of the cornea. Within the cornea, Bowman's capsule is a relatively thin layer (e.g. 20 to 30 μm) that is located below the epithelium, within the anterior one hundred microns of the cornea. The stroma then comprises almost all of the remaining four hundred microns in the cornea. Further, the tissue of Bowman's capsule creates a relatively strong, elastic membrane that effectively resists forces in tension. On the other hand, the stroma comprises relatively weak connective tissue.

Bio-mechanically, Bowman's capsule and the stroma are both significantly influenced by the intraocular pressure that is exerted against the cornea by aqueous in the anterior chamber. In particular, this pressure is transferred from the anterior chamber, and through the stroma, to Bowman's membrane. It is known that how these forces are transmitted through the stroma will affect the shape of the cornea. Thus, by disrupting forces between interconnective tissue in the stroma, the overall force distribution in the cornea can be altered. Consequently, this altered force distribution will then act against Bowman's capsule. In response, the shape of Bowman's capsule is changed, and due to the elasticity and strength of Bowman's capsule, this change will directly influence the shape of the cornea. With this in mind, and as intended for the present invention, refractive surgery is accomplished by making cuts on predetermined surfaces in the stroma to induce a redistribution of bio-mechanical forces that will reshape the cornea.

It is well known that all of the different tissues of the cornea are susceptible to Laser Induced Optical Breakdown (LIOB). Further, it is known that different tissues will respond differently to a laser beam, and that the orientation of tissue being subjected to LIOB may also affect how the tissue reacts to LIOB. With this in mind, the stroma needs to be specifically considered.

The stroma essentially comprises many lamellae that extend substantially parallel to the anterior surface of the eye. In the stroma, the lamellae are bonded together by a glue-like tissue that is inherently weaker than the lamellae themselves. Consequently, LIOB over layers parallel to the lamellae can be performed with less energy (e.g. 0.8 μJ) than the energy required for the LIOB over cuts that are oriented perpendicular to the lamellae (e.g. 1.2 μJ). It will be appreciated by the skilled artisan, however, that these energy levels are only exemplary. If tighter focusing optics can be used, the required energy levels will be appropriately lower. In any event, depending on the desired result, it may be desirable to make only cuts in the stroma. On the other hand, for some procedures it may be more desirable to make a combination of cuts and layers.

In light of the above, it is an object of the present invention to provide methods for performing ophthalmic laser surgery that result in reshaping the cornea to achieve refractive corrections for improvement of a patient's vision. Another object of the present invention is to provide methods for performing ophthalmic laser surgery that require minimal LIOB of stromal tissue. Still another object of the present invention is to provide methods for performing ophthalmic laser surgery that avoid compromising Bowman's capsule and, instead, maintain it intact for use in providing structural support for a reshaped cornea. Another object of the present invention is to provide a system and method for using a single incision to compensate for the adverse effect on vision caused by an asymmetrical optical condition. Yet another object of the present invention is to provide methods for performing ophthalmic laser surgery that are relatively easy to implement and comparatively cost effective.

### SUMMARY OF THE INVENTION

In accordance with the present invention, methods for performing intrastromal ophthalmic laser surgery are provided that cause the cornea to be reshaped under the influence of bio-mechanical forces. Importantly, for these methods, a tissue volume for operation is defined that is located solely within the stroma of the cornea. Specifically, this operational volume extends posteriorly from slightly below Bowman's capsule (membrane) to a substantial depth into the stroma that is equal to approximately nine tenths of the thickness of the cornea. Thus, with the cornea having a thickness “T” (e.g. approximately 500 μm), the operational volume extends from below Bowman's capsule (e.g. 100 μm) to a depth in the cornea that is equal to approximately 0.9 T (e.g. approximately 450 μm). Further, the operational volume extends radially from the visual axis of the eye through a distance of about 5.0 mm (i.e. the operational volume has a diameter of around 10.0 mm).

In general, each method of the present invention requires the use of a laser unit that is capable of generating a so-called femtosecond laser beam. Stated differently, the duration of each pulse in the beam will approximately be less than one picosecond. When generated, this beam is directed and focused onto a series of focal spots in the stroma. The well-known result of this is a Laser Induced Optical Breakdown (LIOB) of stromal tissue at each focal spot. In particular, and as intended for the present invention, movement of the focal spot in the stroma creates a plurality of cuts. Such cuts may include a pattern of radial cuts, or a pattern of radial cuts and cylindrical cuts. Specifically, the radial cuts will be located at a predetermined azimuthal angle θ and will be substantially coplanar with the visual axis of the eye. Each radial cut will be in the operational volume described above and will extend outwardly from the visual axis from an inside radius “rᵢ” to an outside radius “rₒ”. Further, there may be as many “radial cuts” as desired, with each “radial cut” having its own specific azimuthal angle θ.

Geometrically, the cylindrical cuts are made on portions of a respective cylindrical surface. These respective cylindrical surfaces on which cylindrical cuts are made are concentric, and they are centered on the visual axis of the eye. And, they can be circular cylinders or oval (elliptical) cylinders. Further each cylindrical surface has an anterior end and a posterior end. To maintain the location of the cylindrical surface within the operational volume, the posterior end of the cylindrical cut is located no deeper in the stroma than approximately 0.9 T from the anterior surface of the eye. On the other hand, the anterior end of the cylindrical cut is located in the stroma more than at least eight microns in a posterior direction from Bowman's capsule. These cuts will each have a thickness of about two microns.

In a preferred procedure, each cylindrical cut is approximately two hundred microns from an adjacent cut, and the innermost cylindrical cut (i.e. center cylindrical cut) may be located about 1.0 millimeters from the visual axis. There can, of course be many such cylindrical cuts (preferably five), and they can each define a substantially complete cylindrical shaped wall. Such an arrangement may be particularly well suited for the treatment of presbyopia. In a variant of this procedure that would be more appropriate for the treatment of astigmatism, portions of the cylindrical surfaces subjected to LIOB can define diametrically opposed arc segments. In this case each arc segment preferably extends through an arc that is in a range between five degrees and one hundred and sixty degrees. Insofar as the cuts are concerned, each pulse of the laser beam that is used for making the cut has an energy of approximately 1.2 microJoules or, perhaps, less (e.g. 1.0 microJoules).

For additional variations in the methods of the present invention, in addition to or instead of the cuts mentioned above, differently configured layers of LIOB can be created in the stromal tissue of the operational volume. To create these layers, LIOB is performed in all, or portions, of an annular shaped area. Further, each layer will lie in a plane that is substantially perpendicular to the visual axis of the eye. For purposes of the present invention the layers are distanced approximately ten microns from each adjacent layer, and each layer will have an inner diameter “dᵢ”, and an outer diameter “dₒ”. These “layers” will have a thickness of about one micron. As indicated above, the present invention envisions creating a plurality of such layers adjacent to each other, inside the operational volume.

As intended for the present invention, all “cuts” and “layers” (i.e. the radial cuts, cylindrical cuts, and the annular layers) will weaken stromal tissue, and thereby cause a redistribution of bio-mechanical forces in the stroma. Specifically, weaknesses in the stroma that result from the LIOB of “cuts” and “layers” will respectively cause the stroma to “bulge” or “flatten” in response to the intraocular pressure from the anterior chamber. As noted above, however, these changes will be somewhat restrained by Bowman's capsule. The benefit of this restraint is that the integrity of the cornea is maintained. Note: in areas where layers are created, there can be a rebound of the cornea that eventually results in a slight bulge being formed. Regardless, with proper prior planning, the entire cornea can be bio-mechanically reshaped, as desired.

With the above in mind, it is clear the physical consequences of making “cuts” or “layers” in the stroma are somewhat different. Although they will both weaken the stroma, to thereby allow intraocular pressure from aqueous in the anterior chamber to reshape the cornea, “cuts” (i.e. LIOB parallel and radial to the visual axis) will cause the cornea to bulge. On the other hand, “layers” (i.e. LIOB perpendicular to the visual axis) will tend to flatten the cornea. In any event, “cuts,” alone, or a combination of “cuts” with “layers” can be used to reshape the cornea with only an insignificant amount of tissue removal.

In accordance with the present invention, various procedures can be customized to treat identifiable refractive imperfections. Specifically, in addition to cuts alone, the present invention contemplates using various combinations of cuts and layers. In each instance, the selection of cuts, or cuts and layers, will depend on how the cornea needs to be reshaped. Also, in each case it is of utmost importance that the cuts and layers be centered on the visual axis (i.e. there must be centration). Some examples are:

Presbyopia: Cylindrical cuts only need be used for this procedure.

Myopia: A pattern of radial cuts with any cylindrical cuts may be used. If used, the radial cuts are each made with their respective azimuthal angle θ, inside radius “rᵢ” and outside radius “rₒ”, all predetermined. Further, a combination of cylindrical cuts (circular or oval) and annular layers can be used without radial cuts. In this case a plurality of cuts is distanced from the visual axis beginning at a radial distance “r_{c}”, and a plurality of layers is located inside the cuts. Specifically, “dᵢ” of the plurality of layers can be zero (or exceedingly small), and “dₒ” of the plurality of layers can be less than 2r_{c} (d₀<2r_{c}). In an alternative procedure, radial cuts can be employed alone, or in combination with cylindrical cuts and annular layers.

Hyperopia: A combination of cylindrical cuts and annular layers can be used. In this case, the plurality of cuts is distanced from the visual axis in a range between and inner radius “r_{ci}” and an outer radius “r_{co}”, wherein r_{co}>r_{ci}, and further wherein “dᵢ” of the plurality of layers is greater than 2r_{co} (dₒ>dᵢ>2r_{co}).

Astigmatism: Cylindrical cuts can be used alone, or in combination with annular layers. Specifically arc segments of cylindrical cuts are oriented on a predetermined line that is perpendicular to the visual axis. Layers can then be created between the arc segments.

Myopic astigmatism: Cylindrical cuts formed along an arc segment may be used with a pattern of radial cuts. Depending on the required correction, the radial and cylindrical cuts may be intersecting or non-intersecting.

Whenever a combination of cuts and layers are required, the energy for each pulse that is used to create the cylindrical cuts will be approximately 1.2 microJoules. On the other hand, as noted above, the energy for each pulse used to create an annular layer will be approximately 0.8 microJoules.

In another aspect of the present invention, any type of the cuts disclosed above can be selectively used for the purpose of compensating for (i.e. correcting) an asymmetrical optical condition. As envisioned for the present invention, such an asymmetrical optical condition may result for any of several reasons. Further, the cause of the condition may be anatomical, traumatic, or surgically introduced. In the event, there may be a possibility that a single, isolated and uncorrected incision will suffice to counter, minimize or compensate for the adverse, asymmetrical optical condition.

Structurally, a system in accordance with the present invention for performing intrastromal laser refractive surgery on the cornea of an eye to compensate for the adverse effects of an asymmetrical optical condition, includes a laser unit that is electronically connected with a computer. In this combination, the laser unit generates a pulsed femtosecond laser beam, and it focuses the beam to a focal point. Further, the computer is used to operationally configure the laser beam, and to control movement of the laser beam's focal point through tissue of the cornea to produce a Laser Induced Optical Breakdown (LIOB) of the tissue.

Operationally, the computer incorporates a prepared computer program having several intraoperative program sections. For one, there is a program section that provides operational parameters for configuring the laser beam (e.g. spot size, energy and pulse duration). For another, depending on whatever correction or compensation is required, a program section is provided to locate where the compensating incision is to be made, as well as the extent and shape of the incision. A program section is also provided to move the focal spot of the laser beam to perform LIOB.

As envisioned for the specific situation wherein there is an adverse, asymmetrical optical condition, the present invention provides for the use of a single compensating incision. In this case, the single compensating incision will be unique unto itself and will not be connected with another incision. Further, it is envisioned that the compensating incision will, if necessary or desired, extend through the Bowman's membrane.

For purposes of the present invention, the pattern (i.e. extent and shape) of a compensating incision may be a single, unitary surface that is created by a succession of contiguous LIOB spots. Such an incision may be cylindrical, radial or annular as disclosed above. Further, instead of a single, unitary incision, it is envisioned that the pattern for a compensating incision may actually include a plurality of coordinated incisions.

A situation of particular interest for the present invention involves the possibility of a surgically introduced, adverse, asymmetrical optical condition. For example, the creation of a penetration incision in preparation for an integrated cataract surgery may surgically introduce such a condition. Such a penetration incision must be sufficiently extensive to permit the sequential introduction of a phacoemulsification device, an aspiration device, and an inserter of an intraocular lens. Moreover, the penetration incision will be asymmetric with respect to an axis of the eye. Consequently, a compensating incision in accordance with the present invention may be appropriate.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a cross-sectional view of the cornea of an eye shown in relationship to a schematically depicted laser unit;

FIG. 2 is a cross-sectional view of the cornea showing a defined operational volume in accordance with the present invention;

FIG. 3 is a perspective view of a plurality of cylindrical surfaces where laser cuts can be made by LIOB;

FIG. 4 is a cross-sectional view of cuts on the plurality of cylindrical surfaces, as seen along the line 4-4 in FIG. 3, with the cuts shown for a typical treatment of presbyopia;

FIG. 5A is a cross-sectional view of the plurality of cylindrical surfaces as seen along the line 5-5 in FIG. 3 when complete cuts have been made on the cylindrical surfaces;

FIG. 5B is a cross-sectional view of the plurality of cylindrical surfaces as seen along the line 5-5 in FIG. 3 when partial cuts have been made along arc segments on the cylindrical surfaces for the treatment of astigmatism;

FIG. 5C is a cross-sectional view of an alternate embodiment for cuts made similar to those shown in FIG. 5B and for the same purpose;

FIG. 6 is a cross-sectional view of a cornea showing the bio-mechanical consequence of making cuts in the cornea in accordance with the present invention;

FIG. 7 is a perspective view of a plurality of layers produced by LIOB in accordance with the present invention;

FIG. 8 is a cross-sectional view of the layers as seen along the line 8-8 in FIG. 7;

FIG. 9A is a cross-sectional view of a combination of cuts and layers as seen in a plane containing the visual axis of the eye, with the combination arranged for a treatment of hyperopia;

FIG. 9B is a cross-sectional view of a combination of cuts and layers as seen in a plane containing the visual axis of the eye, with the combination arranged for a treatment of myopia;

FIG. 9C is a cross-sectional view of a combination of cuts and layers as seen in a plane containing the visual axis of the eye, with the combination arranged for a treatment of astigmatism;

FIG. 9D is a top plan view of radial cuts that are coplanar with the visual axis;

FIG. 10 is a perspective view of a plurality of cylindrical cuts and a pattern of radial cuts made by LIOB;

FIG. 11A is a cross-sectional view of the plurality of cylindrical cuts and pattern of radial cuts as seen along the line 11-11 in FIG. 10;

FIG. 11B is a cross-sectional view of a plurality of cylindrical cuts and pattern of radial cuts for an alternative embodiment of the present invention;

FIG. 11C is a cross-sectional view of a pattern of radial cuts for another alternative embodiment of the present invention;

FIG. 11D is a cross-sectional view of a pattern of radial cuts for another alternative embodiment of the present invention;

FIG. 11E is a cross-sectional view of a pattern of radial cuts for another alternative embodiment of the present invention;

FIG. 12 is a schematic presentation of an alternate embodiment of the system of the present invention;

FIG. 13A is a top plan view of a cornea with a surgically introduced penetration incision, with a cylindrical cut that is being used as a compensating incision;

FIG. 13B is a top plan view of a cornea with a surgically introduced penetration incision, with a layer cut being used as a compensating incision; and

FIG. 13C is a top plan view of a cornea with a surgically introduced penetration incision, with a radial cut being used as a compensating incision.

### DESCRIPTION OF THE PREFERRED EMBODIMENTS

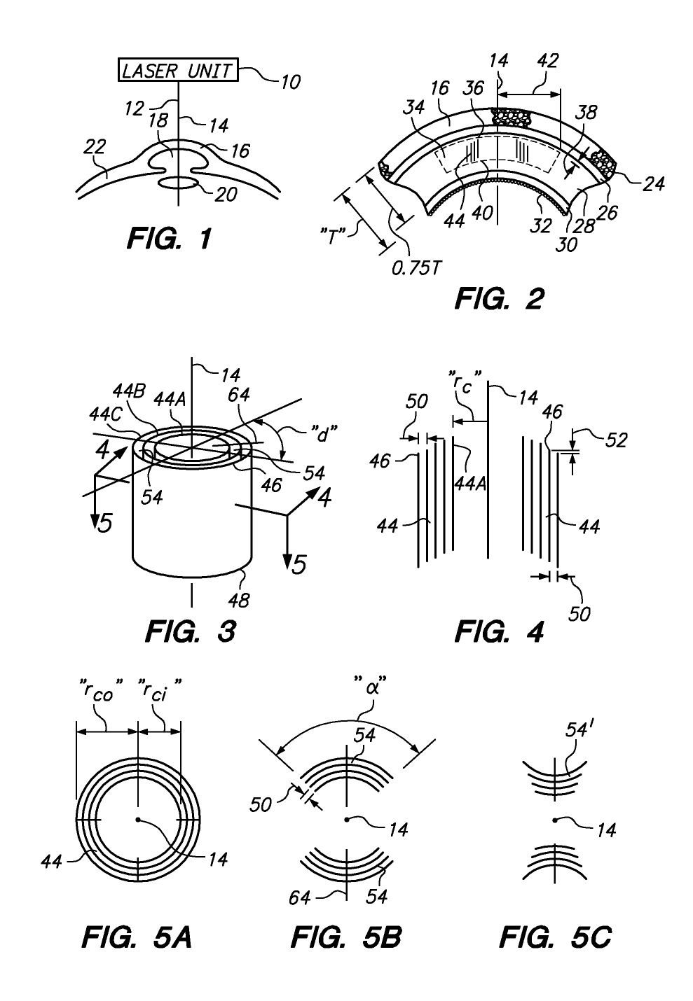

Referring initially to FIG. 1, it will be seen that the present invention includes a laser unit 10 for generating a laser beam 12. More specifically, the laser beam 12 is preferably a pulsed laser beam, and the laser unit 10 generates pulses for the beam 12 that are less than one picosecond in duration (i.e. they are femtosecond pulses). In FIG. 1, the laser beam 12 is shown being directed along the visual axis 14 and onto the cornea 16 of the eye. Also shown in FIG. 1 is the anterior chamber 18 of the eye that is located immediately posterior to the cornea 16. There is also a lens 20 that is located posterior to both the anterior chamber 18 and the sclera 22.

In FIG. 2, five (5) different anatomical tissues of the cornea 16 are shown. The first of these, the epithelium 24 defines the anterior surface of the cornea 16. Behind the epithelium 24, and ordered in a posterior direction along the visual axis 14, are Bowman's capsule (membrane) 26, the stroma 28, Descemet's membrane 30 and the endothelium 32. Of these tissues, Bowman's capsule 26 and the stroma 28 are the most important for the present invention. Specifically, Bowman's capsule 26 is important because it is very elastic and has superior tensile strength. It therefore, contributes significantly to maintaining the general integrity of the cornea 16.

For the methods of the present invention, Bowman's capsule 26 must not be compromised (i.e. weakened). On the other hand, the stroma 28 is intentionally weakened. In this case, the stroma 28 is important because it transfers intraocular pressure from the aqueous in the anterior chamber 18 to Bowman's membrane 26. Any selective weakening of the stroma 28 will therefore alter the force distribution in the stroma 28. Thus, as envisioned by the present invention, LIOB in the stroma 28 can be effectively used to alter the force distribution that is transferred through the stroma 28, with a consequent reshaping of the cornea 16. Bowman's capsule 26 will then provide structure for maintaining a reshaped cornea 16 that will effectively correct refractive imperfections.

While referring now to FIG. 2, it is to be appreciated that an important aspect of the present invention is an operational volume 34 which is defined in the stroma 28. Although the operational volume 34 is shown in cross-section in FIG. 2, this operational volume 34 is actually three-dimensional, and extends from an anterior surface 36 that is located at a distance 38 below Bowman's capsule 26, to a posterior surface 40 that is located at a depth 0.9 T in the cornea 16. Both the anterior surface 36 and the posterior surface 40 essentially conform to the curvature of the stroma 28. Further, the operational volume 34 extends between the surfaces 36 and 40 through a radial distance 42. For a more exact location of the anterior surface 36 of the operational volume, the distance 38 will be greater than about eight microns. Thus, the operational volume 34 will extend from a depth of about one hundred microns in the cornea 16 (i.e. a distance 38 below Bowman's capsule 26) to a depth of about four hundred and fifty microns (i.e. 0.9 T). Further, the radial distance 42 will be approximately 5.0 millimeters.

FIG. 3 illustrates a plurality of cuts 44 envisioned for the present invention. As shown, the cuts 44a, 44b and 44c are only exemplary, as there may be more or fewer cuts 44, depending on the needs of the particular procedure. With this in mind, and for purposes of this disclosure, the plurality will sometimes be collectively referred to as cuts 44.

As shown in FIG. 3, the cuts 44 are made on respective cylindrical surfaces. Although the cuts 44 are shown as circular cylindrical surfaces, these surfaces may be oval. When the cuts 44 are made in the stroma 28, it is absolutely essential they be confined within the operational volume 34. With this in mind, it is envisioned that cuts 44 will be made by a laser process using the laser unit 10. And, that this process will result in Laser Induced Optical Breakdown (LIOB). Further, it is important these cylindrical surfaces be concentric, and that they are centered on an axis (e.g. the visual axis 14). Further, each cut 44 has an anterior end 46 and a posterior end 48. As will be best appreciated by cross-referencing FIG. 3 with FIG. 4, the cuts 44 (i.e. the circular or oval cylindrical surfaces) have a spacing 50 between adjacent cuts 44. Preferably, this spacing 50 is equal to approximately two hundred microns. FIG. 4 also shows that the anterior ends 46 of respective individual cuts 44 can be displaced axially from each other by a distance 52. Typically, this distance 52 will be around ten microns. Further, the innermost cut 44 (e.g. cut 44a shown in FIG. 4) will be at a radial distance “r_{c}” that will be about 1 millimeter from the visual axis 14. From another perspective, FIG. 5A shows the cuts 44 centered on the visual axis 14 to form a plurality of rings. In this other perspective, the cuts 44 collectively establish an inner radius “r_{ci}” and an outer radius “r_{co}”. Preferably, each cut 44 will have a thickness of about two microns, and the energy required to make the cut 44 will be approximately 1.2 microJoules.

As an alternative to the cuts 44 disclosed above, FIG. 3 indicates that only arc segments 54 may be used, if desired. Specifically, in all essential respects, the arc segments 54 are identical with the cuts 44. The exception, however, is that they are confined within diametrically opposed arcs identified in FIGS. 3 and 5B by the angle “α”. More specifically, the result is two sets of diametrically opposed arc segments 54. Preferably, “α” is in a range between five degrees and one hundred and sixty degrees.

An alternate embodiment for the arc segments 54 are the arc segments 54′ shown in FIG. 5C. There it will be seen that the arc segments 54′ like the arc segments 54 are in diametrically opposed sets. The arc segments 54′, however, are centered on respective axes (not shown) that are parallel to each other, and equidistant from the visual axis 14.

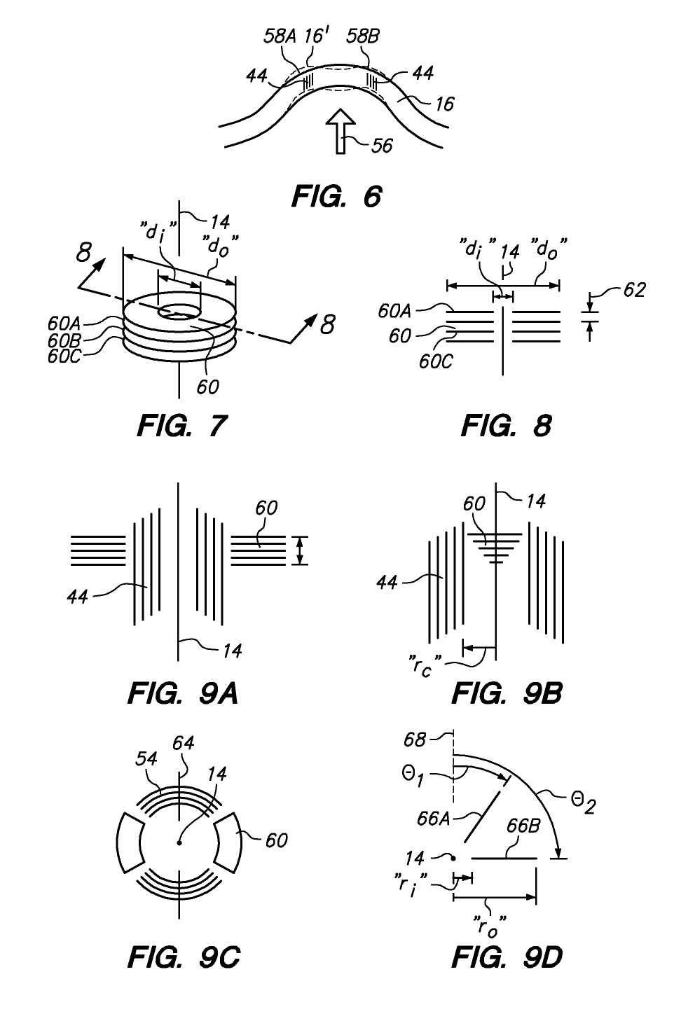

FIG. 6 provides an overview of the bio-mechanical reaction of the cornea 16 when cuts 44 have been made in the operational volume 34 of the stroma 28. As stated above, the cuts 44 are intended to weaken the stroma 28. Consequently, once the cuts 44 have been made, the intraocular pressure (represented by arrow 56) causes a change in the force distribution within the stroma 28. This causes bulges 58a and 58b that result in a change in shape from the original cornea 16 into a new configuration for cornea 16′, represented by the dashed lines. As intended for the present invention, this results in refractive corrections for the cornea 16 that improves vision.

In addition to the cuts 44 disclosed above, the present invention also envisions the creation of a plurality of layers 60 that, in conjunction with the cuts 44, will provide proper vision corrections. More specifically, insofar as the layers 60 are concerned, FIG. 7 shows they are created on substantially flat annular shaped surfaces that collectively have a same inner diameter “dᵢ” and a same outer diameter “dₒ”. It will be appreciated, however, that variations from the configurations shown in FIG. 7 are possible. For example, the inner diameter “dᵢ” may be zero. In that case the layers are disk-shaped. On the other hand, the outer diameter “dₒ” may be as much as 8.0 millimeters. Further, the outer diameter “dₒ” may be varied from layer 60a, to layer 60b, to layer 60c etc.

From a different perspective, FIG. 8 shows that the layers 60 can be stacked with a separation distance 62 between adjacent layers 60 equal to about ten microns. Like the cuts 44 disclosed above, each layer 60 is approximately one micron thick. As mentioned above, the energy for LIOB of the layers 60 will typically be less than the laser energy required to create the cuts 44. In the case of the layers 60 the laser energy for LIOB of the cuts 44 will be approximately 0.8 microJoules.

For purposes of the present invention, various combinations of cuts 44 and layers 60, or cuts 44 only, are envisioned. Specifically, examples can be given for the use of cuts 44 and layers 60 to treat specific situations such as presbyopia, myopia, hyperopia and astigmatism. In detail, for presbyopia, a plurality of only cuts 44 needs to be used for this procedure. Preferably, the cuts 44 are generally arranged as shown in FIGS. 4 and 5A. Further, for presbyopia it is typical for there to be five individual cuts 44 that extend from an inner radius of about 1 mm to an outer radius of about 1.8 mm, with a 200 micron separation between adjacent cuts 44. When hyperopia/presbyopia need to be corrected together, the cuts 44 will then preferably extend further to an outer radius of about 2.3 mm. For hyperopia, a combination of cylindrical cuts 44 and annular layers 60 can be used as shown in FIG. 9A. In this case, the plurality of cuts 44 is distanced from the visual axis 14 in a range between and inner radius “r_{ci}” (e.g. r_{ci}=1 mm) and an outer radius “r_{co}” (e.g. r_{co}=3 mm), wherein r_{co}>r_{ci}, and further wherein “dᵢ” of the plurality of layers 60 is greater than 2r_{co} (dₒ>dᵢ>2r_{co}). For myopia, a combination of cylindrical cuts 44 and annular layers 60 can be used as generally shown in FIG. 9B. In this case a plurality of cuts 44 is distanced from the visual axis 14 beginning at a radial distance “r_{c}”, and a plurality of layers 60, with decreasing outer diameter “dₒ” in a posterior direction, is located inside the cuts 44. More specifically, for this case “dᵢ” of the plurality of layers 60 can be zero (or exceedingly small), and “dₒ” of each layer 60 in the plurality of layers 60 can be less than 2r_{c} (d₀<2r_{c}). And finally, for astigmatism, the portions of cylindrical cuts 44 that form arc segments 54 can be used alone (see FIGS. 5B and 5C), or in combination with annular layers 60 (see FIG. 9C). Specifically arc segments 54 of cylindrical cuts 44 are oriented on a predetermined line 64 that is perpendicular to the visual axis 14. Layers 60 can then be created between the arc segments 54, if desired (see FIG. 9C).

In a variation of the methodologies noted above, the present invention also envisions the creation of radial cuts 66. The radial cuts 66a and 66b shown in FIG. 9D are only exemplary, and are herein sometimes referred to individually or collectively as radial cut(s) 66. Importantly, the radial cuts 66 are coplanar with the visual axis 14, and they are always located within the operational volume 34.

As shown in FIG. 9D, each radial cut 66 is effectively defined by the following parameters: a deepest distance into the stroma 28, Z_{(distal)}, a distance below Bowman's capsule 26, Z₍ₚᵣₒₓᵢₘₐₗ₎, an inner radius, “rᵢ”, an outer radius “rₒ”, and an azimuthal angle “θ” that is measured from a base line 68. By setting values for these parameters, each radial cut 66 can be accurately defined. For example, as shown in FIG. 9D, the radial cut 66a is established by the azimuthal angle θ₁, while the radial cut 66b has an azimuthal angle θ₂. Both of the radial cuts 66a and 66b have the same inner radius “rᵢ” and the same outer radius “rₒ”. The Z_{(distal)} and Z₍ₚᵣₒₓᵢₘₐₗ₎ will be established for the radial cuts 66a and 66b in a similar manner as described above for the cylindrical cuts 44.

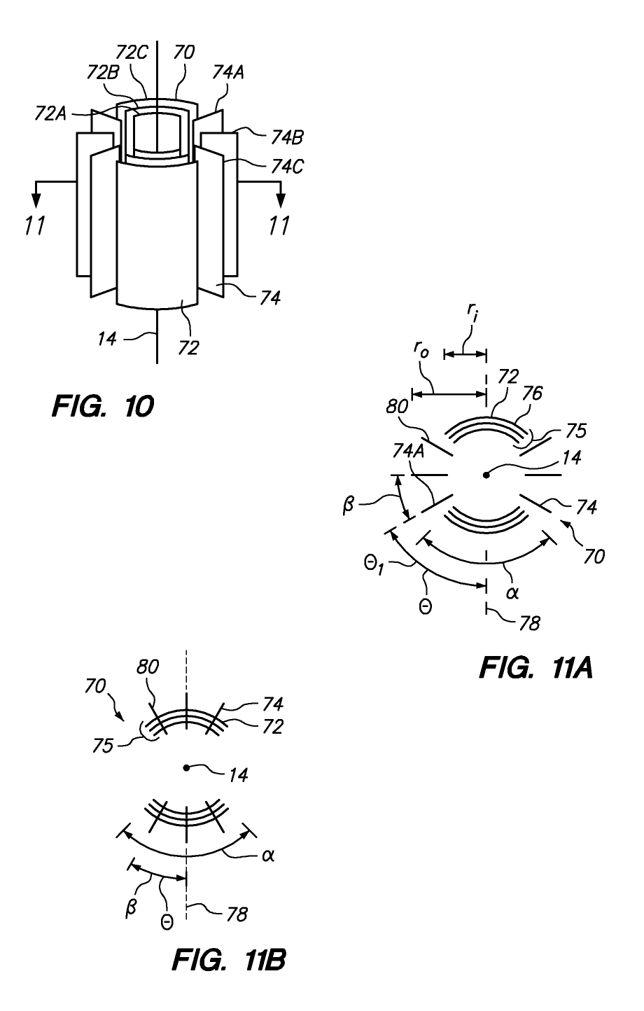

Referring now to FIG. 10, a plurality of cuts 70 is illustrated for an alternate embodiment of the present invention. Specifically, the plurality of cuts 70 shown is intended to correct a myopic astigmatism. As shown, the plurality of cuts 70 includes the cylindrical cuts 72a, 72b, and 72c and the radial cuts 74a, 74b, and 74c. The cylindrical cuts 72a, 72b, and 72c and the radial cuts 74a, 74b, and 74c are only exemplary, as there may be more or fewer cuts 72, 74, depending on the needs of the particular procedure. As shown in FIG. 10, the cylindrical cuts 72 are made on respective cylindrical surfaces. Although the cylindrical cuts 72 are shown as circular cylindrical surfaces, these surfaces may be oval. It is important these cylindrical surfaces be concentric, and that they are centered on an axis (e.g. the visual axis 14).

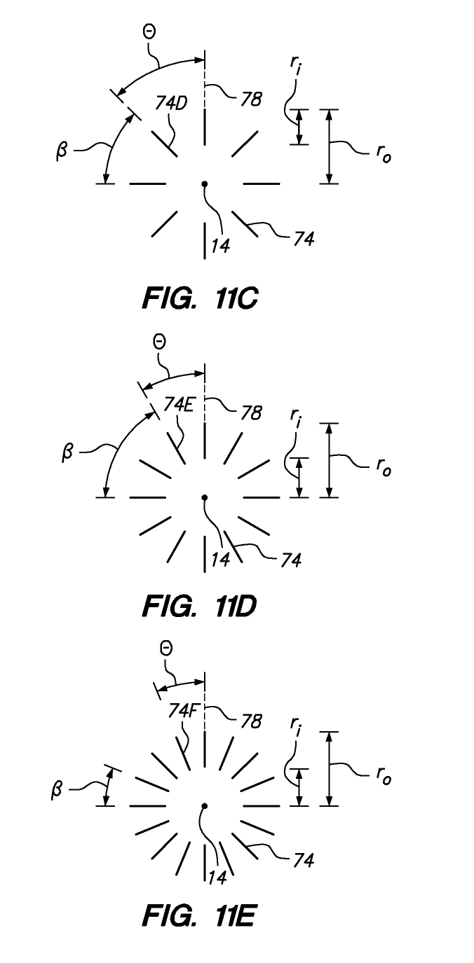

Cross-referencing FIG. 10 with FIG. 11A, it can be seen that the cylindrical cuts 72 are arc segments 76. Specifically, the cylindrical cuts 72 are confined within diametrically opposed arcs identified in FIG. 11A by the angle “α”. More specifically, the result is two sets 75 of diametrically opposed arc segments 76. Preferably, “α” is in a range between five degrees and one hundred and sixty degrees. Further, FIG. 11A shows the cuts 72 centered on the visual axis 14. Preferably, each cut 72 will have a thickness of about two microns, and the energy required to make the cut 72 will be approximately 1.2 microJoules.

As further seen in FIG. 11A, the radial cuts 74 are coplanar with the visual axis 14, and they are always located within the operational volume 34 (shown in FIG. 2). Further, each radial cut 74 is effectively defined by the following parameters: an inner radius, “rᵢ”, an outer radius “rₒ”, and an azimuthal angle “θ” that is measured from a base line 78. By setting values for these parameters, each radial cut 74 can be accurately defined. For example, as shown in FIG. 11A, the radial cut 74a is established by the azimuthal angle θ₁. Each radial cut 74 has the same inner radius “rᵢ” and the same outer radius “rₒ”.

While FIGS. 10 and 11A illustrate a plurality of cylindrical cuts 72 and a pattern of radial cuts 74 that do not intersect, the present invention also envisions intersecting cuts 70. As shown in FIG. 11B, the plurality of cylindrical cuts 72 and the pattern of radial cuts 74 do intersect. In each of the embodiments shown in FIGS. 11A and 11B, the radial cuts 74 can be seen to be comprised in two sets 80 which are diametrically opposed from one another. Within each set 80, the radial cuts 74 are distanced from one another by equal angles β. Likewise, the cylindrical cuts 72 also comprise two diametrically opposed sets 75.

Referring now to FIGS. 11C, 11D, and 11E, a plurality of radial cuts 74 is illustrated for alternate embodiments of the present invention. In FIG. 11C, eight radial cuts 74 are positioned about the visual axis 14. This pattern of radial cuts 74 is intended for a myopic correction of −0.75 diopters. In FIG. 11D, twelve radial cuts 74 are positioned about the visual axis 14. This pattern of radial cuts 74 is intended for a myopic correction of −1.25 diopters. In FIG. 11E, sixteen radial cuts 74 are positioned about the visual axis 14. This pattern of radial cuts 74 is intended for a myopic correction of −2.0 diopters.

As shown in FIGS. 11C, 11D, and 11E, each radial cut 74 is coplanar with the visual axis 14, and located within the operational volume 34 (shown in FIG. 2). Further, each radial cut 74 is effectively defined by the following parameters: an inner radius, “rᵢ”, an outer radius “rₒ”, and an azimuthal angle “θ” that is measured from a base line 78. By setting values for these parameters, each radial cut 74 can be accurately defined. For example, as shown in FIG. 11C, the radial cut 74d is established by the azimuthal angle θ. In FIG. 11D, the radial cut 74e is established by the azimuthal angle θ. Further, in FIG. 11E, the radial cut 74f is established by the azimuthal angle θ.

In FIGS. 11C, 11D, and 11E, each radial cut 74 has the same inner radius “rᵢ” and the same outer radius “rₒ”. In FIG. 11C, each radial cut 74 is distanced from the adjacent radial cut 74 by angle β equal to 45 degrees. Further, in FIG. 11D, each radial cut is distanced from the adjacent radial cut 74 by angle β equal to 30 degrees. In FIG. 11E, each radial cut is distanced from the adjacent radial cut 74 by angle β equal to 22.5 degrees.

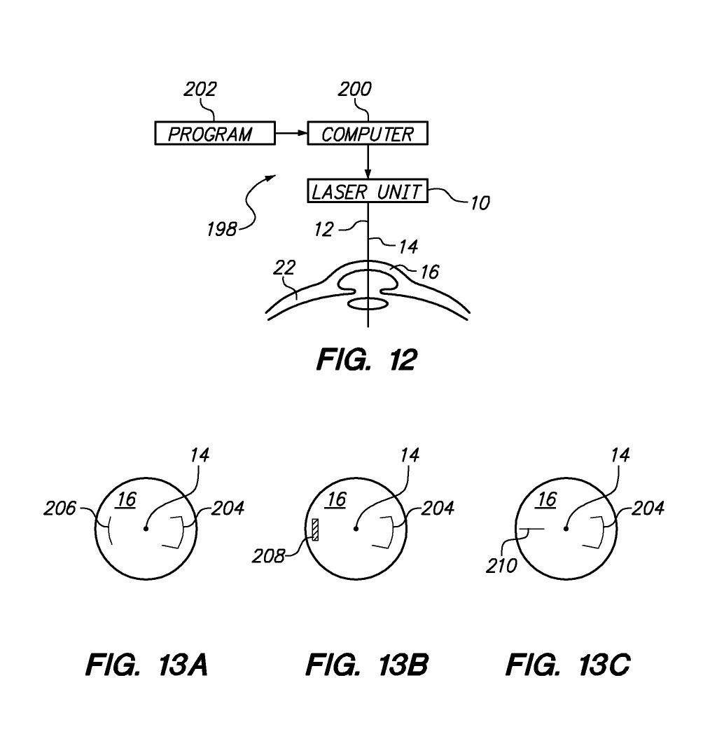

Referring now to FIG. 12, a system for performing intrastromal laser refractive surgery on a cornea 16 of an eye is shown and is generally designated 198. As shown, the system 198 incorporates a computer 200 for controlling the laser unit 10, and FIG. 12 further indicates that the computer 200 will control the laser unit 10 in accordance with input from a computer program 202. In this combination, the system 198 is employed to correct, or compensate, for the adverse effect of an asymmetrical optical condition in the stroma 28. As envisioned for the present invention, the cause of such an optical condition may be anatomical, traumatic, or surgically introduced. For purposes of disclosure here, the latter case (i.e. a surgically introduced condition) is considered in detail.

As a general proposition, an adverse, asymmetrical, optical condition that is externally introduced into the cornea 16, will most likely result from a surgical operation. For instance, a penetration incision 204 (see FIGS. 13A-C) that is necessarily made preparatory to an integrated cataract surgery may cause such a condition. In any event, and regardless of the actual cause of the adverse optical condition, the system 198 can be employed to eliminate or minimize the condition by creating a compensating (i.e. correcting) incision. Examples of various compensating incisions envisioned by the present invention are shown in FIGS. 13A, 13B and 13C. Respectively, these compensating incisions are: an arcuate cut (compensating incision 206, FIG. 13A), an annular cut (compensating incision 208, FIG. 13B), and a radial cut (compensating incision 210, FIG. 13C).

In FIGS. 13A, 13B and 13C it will be seen that each of the compensating incisions 206/208/210 is singularly unique, and each one is based on a predetermined pattern. For the present invention, the specific pattern used for a compensating incision 206/208/210 depends on the nature of the particular optical condition that is to be corrected. Further, although the incisions 206/208/210 shown in FIGS. 13A-C are individually separate and distinct, additional compensating incisions of a same or different pattern (not shown) may be collectively employed, as needed. If used in a plurality, however, each of the compensating incisions 206/208/210 will still be disconnected from each other. Stated differently, each compensating incision 206/208/210 is individually unique.

For an operation of the system 198, a computer program 202 is inputted into the computer 200 to control the laser unit 10. Specifically, the computer program 202 will control the laser unit 10 to perform intrastromal laser refractive surgery on the stroma 28 of the cornea 16. To do this, a section of the program 202 is provided to configure the laser beam 12 of the laser unit 10 in a manner that causes Laser Induced Optical Breakdown (LIOB) of tissue in the cornea 16. Specifically, for this configuration, the computer program 202 incorporates operational parameters that will set a pulse repetition rate, establish a duration of less than one picosecond per pulse, and determine a pulse energy level. The object here is to thereby weaken the cornea 16 for a redistribution of biomechanical forces in the stroma 28 that corrects a predetermined asymmetrical optical condition of the eye.

Another section of the program 202 is also provided for moving the focal spot of the laser beam 12 through the cornea 16 in a pattern of successive focal spots. The result here is the creation of at least one, singularly unique, compensating incision 206/208/210 in the stroma 28. In the event, the compensating incision 206/208/210 will be defined by the pattern that is established in the computer program 202, and the compensating incision 206/208/210 will be made relative to the axis 14 to counter and minimize the adverse effect on vision caused by the asymmetrical optical condition.

While the particular System and Method for Using Compensating Incisions in Intrastromal Refractive Surgery as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for performing intrastromal laser refractive surgery on the cornea of an eye which comprises:
a laser unit for generating a pulsed laser beam to provide for Laser Induced Optical Breakdown (LIOB) of tissue, to weaken stromal tissue and cause a redistribution of biomechanical forces in the stroma for correcting a predetermined asymmetrical optical condition of the eye; and
a computer electronically connected to the laser unit for controlling the laser unit, to surgically reshape the cornea by creating at least one singularly unique compensating incision in the stroma, wherein the compensating incision is defined by a pattern to counter and minimize the adverse effect on vision caused by the asymmetrical optical condition.

2. A system as recited in claim 1 further comprising a computer program, wherein the eye defines an axis and the computer program comprises:
a program section for configuring the laser beam to include pulses of less than one picosecond duration, and for directing the laser beam to a focal spot;
a program section for positioning the pattern of the compensating incision in the stroma; and
a program section for moving the focal spot of the laser beam over the pattern relative to the axis to perform LIOB to compensate for the asymmetrical optical condition.

3. A system as recited in claim 1 wherein the pattern includes a plurality of disconnected cuts.

4. A system as recited in claim 1 wherein the pattern is an arcuate cut.

5. A system as recited in claim 1 wherein the pattern is an annular cut.

6. A system as recited in claim 1 wherein the pattern is a radial cut.

7. A system as recited in claim 1 wherein the predetermined asymmetrical optical condition is surgically created.

8. A system as recited in claim 7 wherein the predetermined asymmetrical optical condition is a penetration incision created in preparation for an integrated cataract surgery.

9. A computer program for use with a computer to control a laser unit for performing intrastromal laser refractive surgery on the cornea of an eye, wherein the eye defines an axis and the computer program comprises:
a program section for configuring a laser beam of the laser unit to cause Laser Induced Optical Breakdown (LIOB) of tissue, and to weaken the tissue for a redistribution of biomechanical forces in the stroma for correction of a predetermined asymmetrical optical condition of the eye; and
a program section for moving the focal spot of the laser beam in a pattern of successive focal spots to create at least one singularly unique compensating incision in the stroma, wherein the compensating incision is defined by the pattern and is made relative to the axis to counter and minimize the adverse effect on vision caused by the asymmetrical optical condition.

10. A computer program as recited in claim 9 wherein the pattern includes a plurality of disconnected cuts.

11. A computer program as recited in claim 9 wherein configuration of the laser beam involves setting a pulse repetition rate, establishing a pulse duration of less than one picosecond, and determining a pulse energy level.

12. A computer program as recited in claim 9 wherein the pattern is an arcuate cut.

13. A computer program as recited in claim 9 wherein the pattern is an annular cut.

14. A computer program as recited in claim 9 wherein the pattern is a radial cut.

15. A computer program as recited in claim 9 wherein the predetermined asymmetrical optical condition is surgically created.

16. A computer program as recited in claim 15 wherein the predetermined asymmetrical optical condition is a penetration incision created in preparation for an integrated cataract surgery.

17. A method for performing intrastromal laser refractive surgery on the cornea of an eye, wherein the eye defines an axis and the method comprises the steps of:
configuring a laser beam of the laser unit to cause Laser Induced Optical Breakdown (LIOB) of tissue, and to weaken the tissue for a redistribution of biomechanical forces in the stroma for correction of a predetermined asymmetrical optical condition of the eye; and
moving the focal spot of the laser beam in a pattern of successive focal spots to create at least one singularly unique compensating incision in the stroma, wherein the compensating incision is defined by the pattern and is made relative to the axis to counter and minimize the adverse effect on vision caused by the asymmetrical optical condition.

18. A method as recited in claim 17 further comprising the steps of:
setting a pulse repetition rate;
establishing a pulse duration of less than one picosecond; and
determining a pulse energy level.

19. A method as recited in claim 17 wherein the pattern includes a plurality of disconnected cuts.

20. A method as recited in claim 17 wherein the pattern is selected from a group consisting of an arcuate cut, an annular cut, and a radial cut.
